(19) United States
(12) Patent Application Publication
(10) Pub. No.: US 2008/0249119 A1
(43) Pub. Date: Oct. 9, 2008

(54) Homogemcitabines

(75) Inventors: Christian R. Noe, Wien (AT); Muhamed Jasic, Wien (AT); Hermann Kollmann, Linz (AT); Karmin Saadat, Wien (AT)

Correspondence Address: FULBRIGHT & JAWORSKI L.L.P., 600 CONGRESS AVE., SUITE 2400, AUSTIN, TX, 78701, US

(73) Assignee: PHARMACON-FORSCHUNG UND BERATUNG GMBH, Brunnenthal (AT)

(21) Appl. No.: 11/996,203

(22) PCT Filed: Jul. 20, 2006
(86) PCT No.: PCT/AT06/00308

§ 371 Date: Jan. 18, 2008

(30) Foreign Application Priority Data
Jul. 20, 2005 (AT) A 1224/2005

(51) Int. Cl. A61K 31/513; C07D 405/04; C07D 307/20; A61P 35/00

(52) U.S. Cl. 514/274; 544/317; 549/478

(57) ABSTRACT

The invention relates to homogemcitabines of general formula (2), with the meanings for the substituents as given in claim 1, method for production thereof, use for the production of the active agent gemcitabin and the use thereof for the production of medicaments for the treatment of proliferative diseases. The invention further relates to novel intermediates, as used in the inventive method.

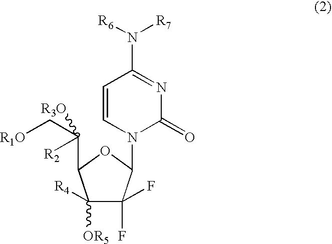

The present invention relates to homogemcitabines, methods for their production, their use for producing the active substance gemcitabine and their use for producing medicaments for treatment of proliferative diseases. Furthermore, the present invention relates to novel intermediates as used in the inventive method.

Meanwhile, the active substance gemcitabine of the formula 1

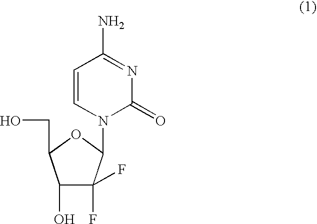

has been successfully established as antimetabolite for treatment of cancer diseases. Since production of this nucleoside twice flourized in the sugar moiety involves a considerable synthetic effort, many studies have meanwhile become known which primarily deal with improvement of the originally published synthesis that starts from protected glycerin aldehyde. The key step in this synthesis is the nucleosidation of a fluoridated pentose derivative with the activated nucleobase cytosine.

A first subject-matter of the present invention are homologs of gemcitabine of the general formula 2

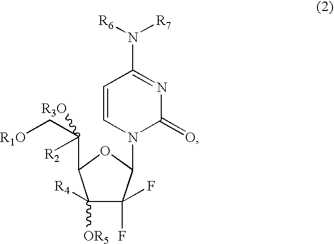

wherein R₁, R₃ and R₅ represent hydrogen or a suitable hydroxy protective group, as known in the prior art, e.g. from “Protective Groups in Organic Synthesis” (Green, Wuts), 3^{rd} edition, John Wiley & Sons, Inc., pages 17 to 245, in particular represent a benzoyl group, each of R₂ and R₄ represents hydrogen or alkyl with 1 to 6 C-atoms, and each of R₆ and R₇ represents hydrogen or a suitable amino protective group, as known in the prior art, e.g. from “Protective Groups in Organic Synthesis” (Green, Wuts), 3^{rd} edition, John Wiley & Sons, Inc., pages 494 to 653, in particular represents acetyl, alkylsilanyl or arylalkyl-silanyl with 1 to 6 C-atoms in the alkyl moiety, and the wavy line in each case represents both possible configurations of —OR₃ and/or —OR₅ with regard to the parent substance.

Compounds of the general formula 2, in particular where each of R₁ to R₇ represents hydrogen, are interesting candidates as therapeutic agents for proliferative diseases, wherein the use of homogemcitabines of the general formula 2, for producing medicaments for treatment of proliferative diseases, constitutes a further subject matter of the present invention. In particular, it is assumed that compounds of the general formula 2 for producing medicaments for treatment of NSCLC (non-small cell lung cancer), mamma carcinoma, ovarian carcinoma, pancreas carcinoma and bladder carcinoma are suitable, namely individually or in combination with other active substances/medicaments. The established therapeutic models usually provide for a combination of different cytostatics, e.g. in the case of bladder carcinoma there is a combination with cisplatin, in the case of ovarian carcinoma with carboplatin.

A further subject matter of the present invention is a method for producing such homologs of gemcitabine of the general formula 2. The compounds can be produced, for example, by nucleosidation of suitable fluoridated and, optionally, protected hexoses of the general formula 3

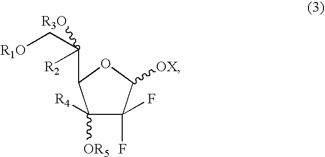

wherein R₁ to R₅ are as defined above, X represents hydrogen or an activating group known per se, preferably an alkylsulphonyl residue with 1 to 6 C-atoms in the alkyl moiety, and the wavy line in each case represents both possible configurations of OX, —OR₃ and/or —OR₅ with regard to the parent substance,

with a cytosine, provided with protective groups, of the general formula 4

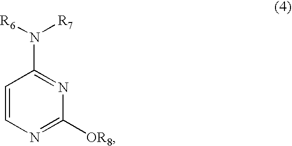

wherein R₆ and R₇ are as defined above, wherein, preferably, at least one of the two groups represents trialkylsilanyl or triarylalkyl-silanyl each having 1 to 6 C-atoms in the alkyl moiety and R₈ is a suitable leaving group, preferably is equal to R₆ and R₇, wherein, subsequently, any protective groups possibly still present are optionally cleaved to obtain compounds of the general formula (2), wherein each of R₁ to R₇ represents hydrogen.

The above-mentioned fluoridated hexoses of the general formula 3 are novel compounds and are also a further subject matter of the present invention. They, in turn, can be produced by adding a defluoridated component to an enantiomerically-pure structural C4-element which, with one of its chirality centres, determines the D-configuration according to the carbohydrate nomenclature, preferably by adding a defluorinated acetic-acid derivative of the general formula 5

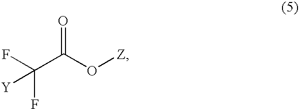

wherein Y represents a suitable leaving group, such as bromine, chlorine or iodine, or hydrogen, und Z is an alkyl with 1 to 6 C-atoms, to a protected derivative of an L-threose or a D-erythrose of the general formula 6

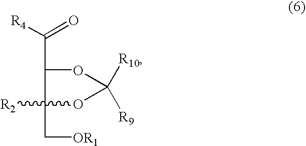

wherein R₁, R₂ and R₄ are defined as above, and each of R₉ and R₁₀ represents hydrogen, an alkyl group with 1 to 3 C-atoms or phenyl.

The addition product is then cyclized to the lactone of the general formula 3

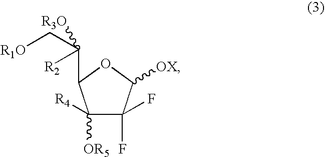

wherein R₁, R₂, R₄ and R₅ are as defined above, R₃ represents hydrogen, and the wavy line, together with OX, represents a keto group.

For producing homogemcitabines, free present hydroxyl groups of the compounds of the general formula 3 are then once more protected by using a suitable hydroxy protective group, as known in the prior art, e.g. from “Protective Groups in Organic Synthesis” (Green, Wuts), 3^{rd} edition, John Wiley & Sons, Inc., pages 17 to 245, preferably with an optionally substituted benzoyl group. The protected lactone is hydrogenated, e.g. by means of a complex hydride, and is, thus, converted to the lactole of the general formula 3, wherein each of R₁, R₃ and R₅ represents suitable hydroxy protective groups, preferably optionally substituted benzoyl groups, and X represents hydrogen, which lactole is activated for the nucleosidation reaction following thereupon, wherein the activation is preferably effected by introducing an alkylsulphonyl residue with 1 to 6 C-atoms in the alkyl moiety. The activated lactole is then reacted with a cytosine, provided with protective groups, of the general formula 4, any protective groups possibly still present are thereafter optionally cleaved to obtain compounds of the general formula (2), wherein each of R₁ to R₇ represents hydrogen.

In particular, the novel compounds of the general formula 3 are also well-suited as starting product for producing gemcitabine. To this end, as already described, the compound of the general formula 3 is nucleodized to a compound of the general formula 2 with a cytosine, provided with protective groups, of the general formula 4, the protective groups on the hydroxyl groups of the sugar moiety and on the cytosine are optionally removed and a compound of the general formula 2

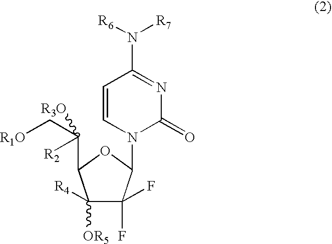

is obtained, wherein each of R₁ to R₇ represents hydrogen, and the wavy line in each case represents both possible configurations of —OR₃ and/or —OR₅ with regard to the parent substance, a glycol cleavage to the aldehyde of the formula 7

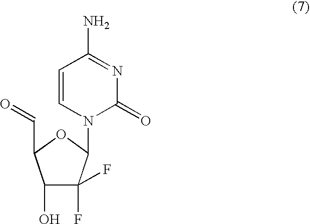

is then performed on the compound, wherein with respect to the glycol cleavage there can be used both the pure β-anomer of compounds of the general formula 2, wherein each of R₁ to R₇ represents hydrogen, and the mixture of α- and β-anomers which is formed during nucleosidation. In this context, the glycol cleavage is performed by means of conventional reagents, preferably by means of a periodate, whereupon the aldehyde group of the aldehyde 7 will be reduced to the hydroxy group with a complex hydride, preferably with sodium borohydride. The reduction is performed best in a one-pot reaction after glycol cleavage, whereby gemcitabine of the formula 1

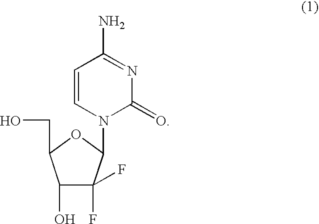

is obtained directly and can be converted into a product of pharmaceutical quality by recrystallization.

A further subject matter of the present invention is a method for producing compounds of the general formula 2 by fluoridation of ketohexose nucleosides of the general formula 12

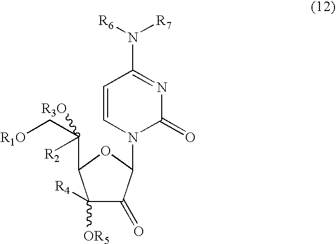

with a suitable fluorinating agent, preferably with DAST (diethylaminosulfur trichloride) in combination with HF.

The ketohexose nucleosides of the general formula 12 are also novel compounds and represent a yet further subject matter of the present invention. They can be produced in that a compound of the general formula 8

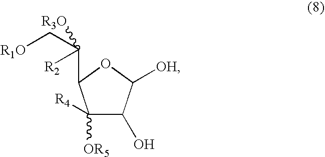

wherein R₁, R₃ and R₅ represent a suitable hydroxy protective group, as they are known in the prior art, e.g. from “Protective Groups in Organic Synthesis” (Green, Wuts), 3^{rd} edition, John Wiley & Sons, Inc., pages 17 to 245, in particular represent a benzoyl or acetyl group, each of R₂ and R₄ represents hydrogen or alkyl with 1 to 6 C-atoms, and the wavy line in each case represents both possible configurations of —OR₃ and/or —OR₅ with regard to the parent substance, is provided, in positions 1 and 2, with protective groups known per se and suitable for activation of the lactole group, preferably with phenoxyacetyl, acetyl or benzoyl groups, whereby the lactole group of the thus obtained compound of the general formula 9

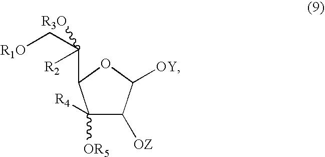

wherein R₁ to R₅ are as defined in formula 8, and each of X and Y represents a protective group known per se and suitable for activation of the lactole group, preferably a phenoxyacetyl, acetyl or benzoyl group, is activated for nucleosidation. Thereafter, the compound of the general formula 9 is reacted with a protected cytosine derivative of the general formula 4 as initially described, obtaining a compound of the general formula 10

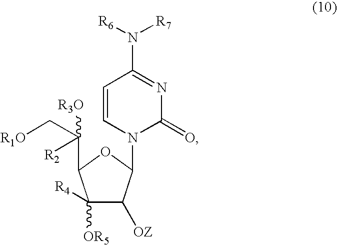

wherein the respective substituents are as defined in formula 9 and formula 4, respectively. The reaction can be performed analogous to the nucleosidation already mentioned above. Subsequently, one essential step consists in the selective cleavage of the protective and/or activating group in position 2 of the compound of the general formula 10, which is preferably performed as hydrazinolysis, obtaining a compound of the general formula 11

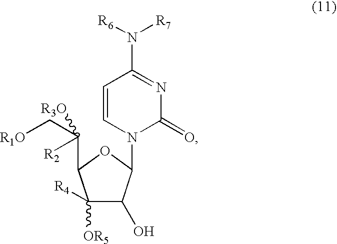

wherein the substituents are as defined in formula 10. By oxidation of the hydroxyl group in position 2 of the compound of the general formula 11, which can be done using different known methods, such as oxidation with chromium compounds or Swern-reaction, preferably, however, using the catalyst TEMPO, a compound of the general formula 12

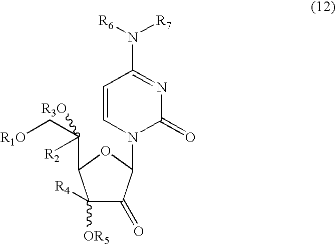

is obtained, wherein the substituents are as defined in formula 10. Subsequently, difluoridation of the compound of the general formula 12 is effected, e.g. by DAST to the compound of the general formula 2

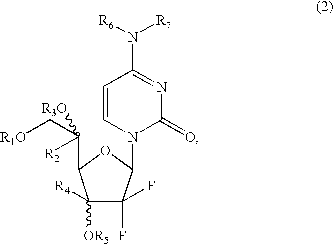

wherein the substituents, optionally after removal of the protective groups on the hydroxyl groups of the sugar moiety and on the cytosine residue, are as initially defined in formula 2. It goes without saying that also the thus obtained compound of the general formula 2, as already aforementioned, can be converted into gemcitabine of the formula 1

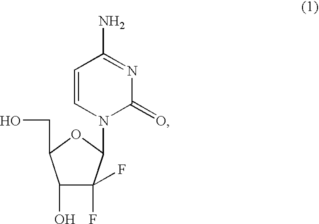

whereupon gemcitabine of pharmaceutical quality will be obtained by recrystallization.

The present invention will now be explained in more detail by way of the following examples. In the enclosed drawings

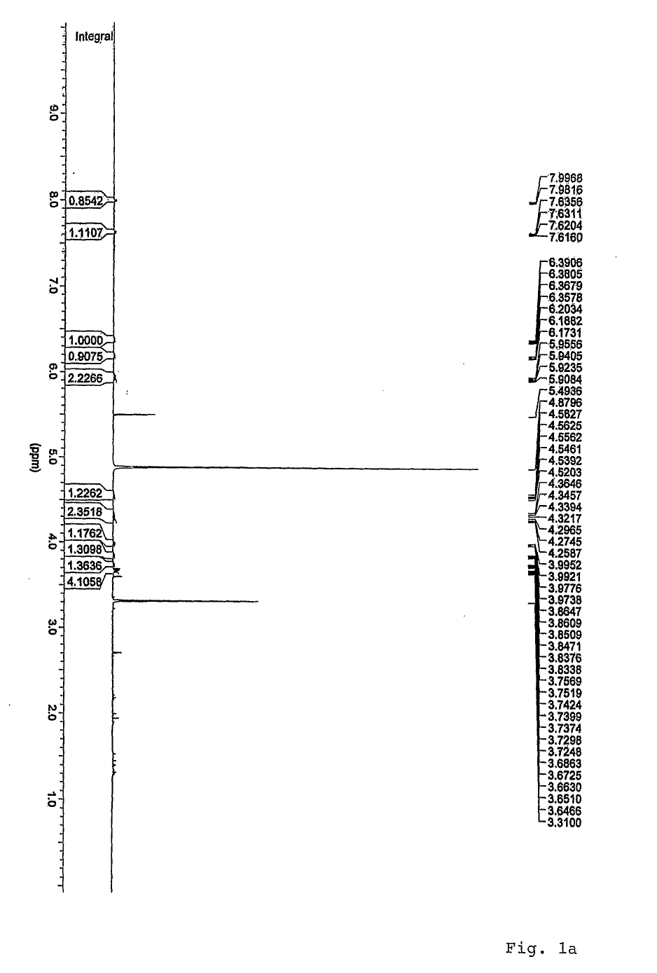

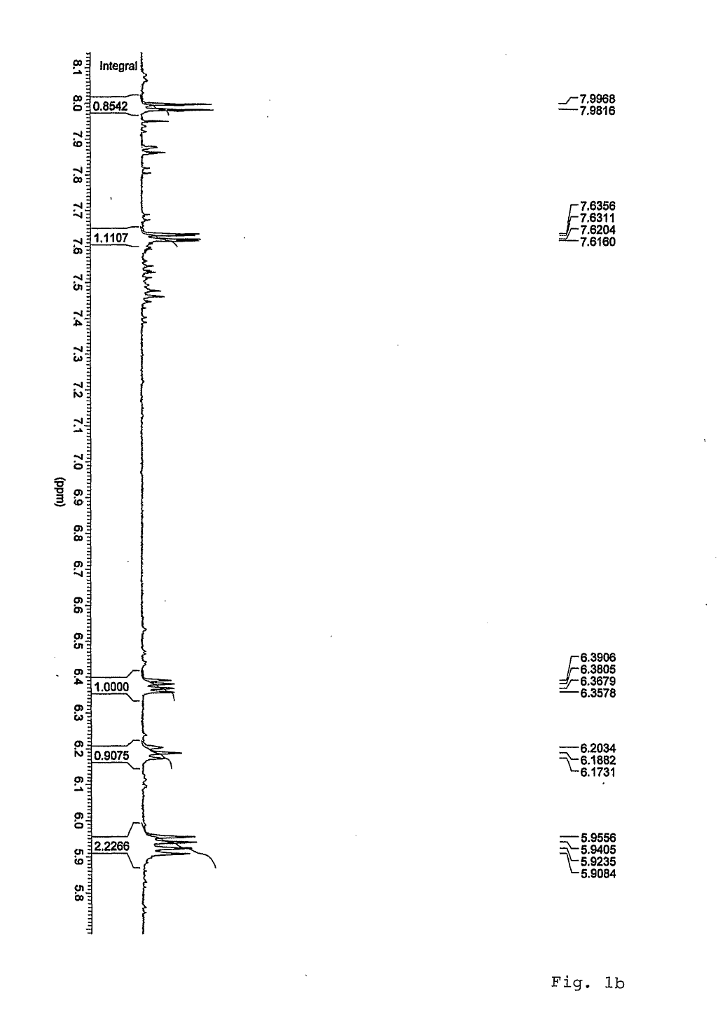

FIGS. 1a, 1b and 1c show the ¹H-NMR-spectra of the anomer mixture of compound (XIII), of a homogemcitabine of the general formula 2, wherein R₁ to R₆ represent hydrogen, and

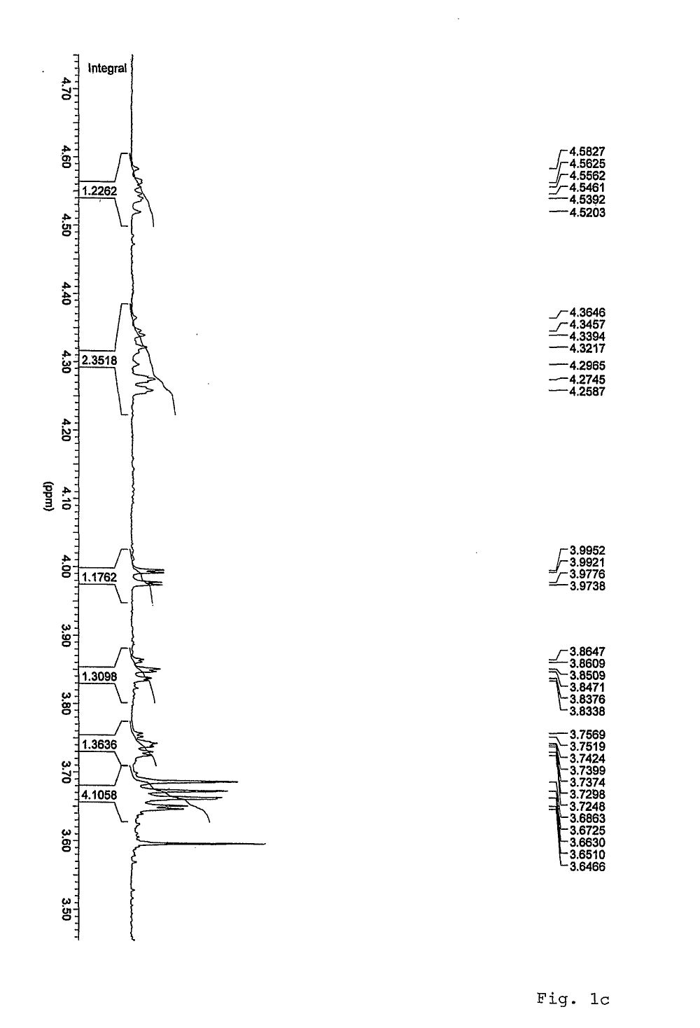

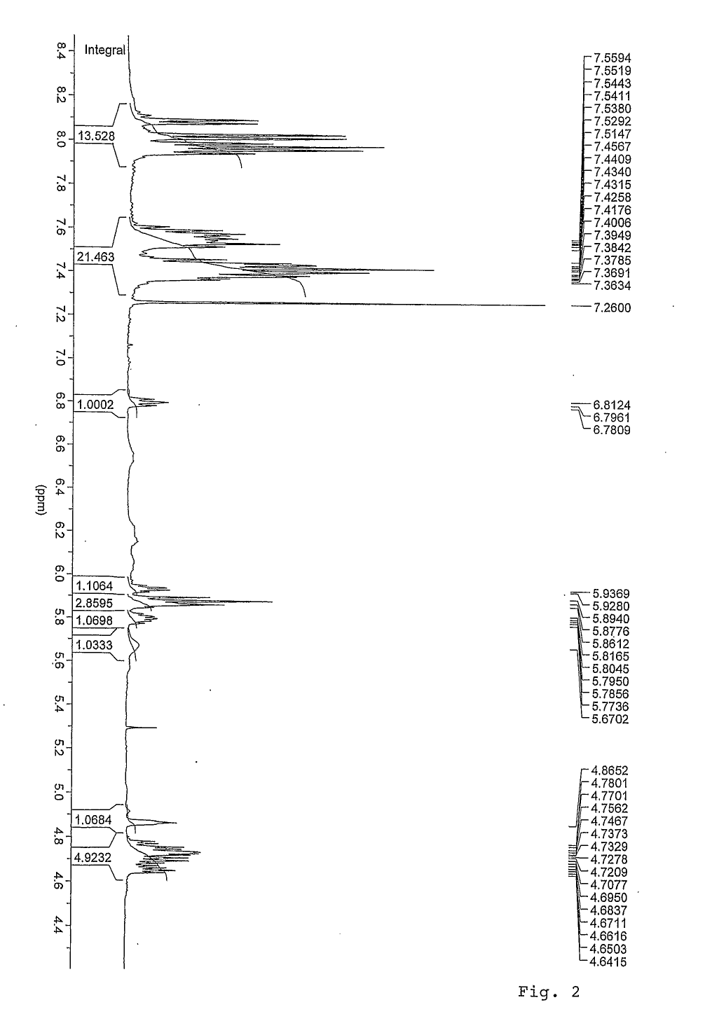

FIG. 2 shows the ¹H-NMR-spectrum of the anomer mixture of compound (XII), a compound of the general formula 2, wherein R₁, R₃ and R₅ represent a benzoyl group und each of R₂ and R₄ is hydrogen.

### EXAMPLE 1

### 2,2-dimethyl-1,3-dioxolane-4,5-dimethanol-4-benzoate (V)

At 0° C. 14.76 g of benzoyl chloride are quickly added to a solution of 17 g of 2,2-Dimentyl-1,3-dioxolane-4,5-dimethanol in 34 ml pyridine. After 2 hours of stirring under cooling, MTBE and H₂O are added to the reaction mixture. The phases are separated, the aqueous phase is extracted with MTBE. The combined organic phases are washed with semi-saturated NaHCO₃-solution, dried and evaporated. The remainder is separated by means of VFC (vacuum-flash chromatography) over silica gel with toluene/EtOAc (10+1), thereafter (1+1). 13.7 g of monobenzoate and 10.5 g of dibenzoate are obtained.

2,2-Dimethyl-1,3-dioxolane-4,5-dimethanol-4-monobenzoate: Bp: 113-117° C. (0.05 mm of Hg).

¹H-NMR: (CDCl₃): δ (ppm)=8.05-7.38 (m, 5H, Ar—H), 4.55-4.37 (m, 2H, CH₂), 4.29-4.20 (m, 1H, H-3), 4.08-4.00 (m, 1H, H-2), 3.84-3.74 (dd, 2H, CH₂OH), 2.47-2.32 (s, 1H, OH), 1.43 (s, 6H, 2×CH₃).

2,2-Dimethyl-1,3-dioxolane-4,5-dimethanol-4,5-dibenzoate; Mp: 85-86° C.; ¹H-NMR: (CDCl₃): δ(ppm): 8.08-7.39 (m, 10H, Ar—H), 4.63-4.51 (m, 4H, 2×CH₂), 4.38-4.28 (m, 2H, H-2, H-3), 1.48 (s, 6H, 2×CH₃);

### EXAMPLE 2

### 5-Benzoxymethyl-2,2-dimethyl-1,3-dioxolane-4-carbaldehyde (VI)

### a) By Means of Swern-Oxidation

At −70° C. 1.76 g of DSMO in 10 ml of dichloromethane are added to a solution of 1.86 g oxalyl chloride in 13 ml of anhydrous dichloromethane such that the temperature remains below −60° C. After 15 min a solution of 8.3 g of 2,2-Dimethyl-1,3-dioxolane-4,5-dimethanol-4-benzoate (V) in 25 ml of anhydrous dichloromethane is added at the same temperature, and the solution is stirred for 1 hour at −70° C. 8.52 ml of triethylamine/dichloromethane (1:1) are added at a temperature of below −60° C. After 15 min cooling means is removed and 40 ml of H₂O are added to the reaction mixture after room temperature has been reached. The phases are separated, the aqueous phase is extracted with dichloromethane. The combined organic phases are washed with 1N HCl, 5% NaHCO₃-solution and saturated NaC1-solution, dried and evaporated. 2.73 g (91.7%) of 5-Benzoxymethyl-2,2-dimethyl-1,3-dioxolane-4-carbaldehyde are obtained as a yellow oil.

Bp.: 107-109° C. (0.05 mm of Hg).

¹H-NMR: (CDCl₃): δ (ppm)=9.83-9.82 (s, 1H, HC═O), 8.06-7.26 (m, 5H, Ar—H), 4.60-4.56 (m, 2H, CH₂), 4.47-4.34 (m, 1H, CH), 4.33 (m, 1H, CH), 1.51-1.49 (s, 3H, CH₃), 1.45-142 (s, 3H, CH₃).

### b) By Means of Chromium Oxidation

5.6 g of pyridine and 2.6 g of chromium trioxide are added to a solution of 1.5 g of 2,2-Dimethyl-1,3-dioxolane-4,5-dimethanol-4-benzoate (V) in dry dichloromethane. After 3 hours of stirring at RT, the reaction mixture is diluted with MTBE, filtrated over celite, evaporated and dried. 0.7 g of 5-Benzoxymethyl-2,2-dimethyl-1,3-dioxolane-4-carbaldehyde are obtained as a light-yellow oil.

### EXAMPLE 3

### 5-Benzoxymethyl-α,α-difluoro-β-hydroxy-2,2-dimethyl-4-[1,3]-dioxolane-propanoic-acid-ethyl-ester (VII)

4.2 g of activated zinc dust are added to a solution of 18.9 g of 5-Benzoxymethyl-2,2-dimethyl-1,3-dioxolane-4-carbaldehyde and 13 g of bromodifluoro acetic acid ethyl ester in 240 ml of anhydrous THF/diethylether (1:1). The reaction mixture is heated to reflux temperature in an ultrasonic bath for 4 hours. Subsequently, 240 ml of 0.5N HCl are added and the aqueous phase is extracted with MTBE three times. The organic phases collected are washed with 5% NaHCO₃ solution, dried over Na₂SO₄, filtrated and evaporated. 7.85 g of oily product are obtained.

¹H-NMR: (CDCl₃): δ (ppm)=8.07-7.42 (m, 5H, Ar—H), 4.69-4.67/4.50-4.62 (m, 2H, CH₂), 4.44-4.41 (m, 1H, CH), 4.40-4.23 (m, 3H, CH₂, CH), 4.09-4.05 (t, 1H, CH), 1.40-1.38 (d, 6H, 2×CH₃), 1.37-134 (m, 3H, CH₃).

### EXAMPLE 4

### 1-(4,4-Difluoro-tetrahydro-3-hydroxy-5-oxo-2-furyl)-1,2-ethanediol-2-benzoate (VIII)

1.5 g of H₂O and 0.4 g of TFA are added to a solution of 5.9 g of 5-Benzoxymethyl-α,α-difluoro-β-hydroxy-2,2-dimethyl-4-[1,3]-dioxolane-propanoic-acid-ethyl-ester (VII) in 30 ml of acetonitrile, and the solution is heated under reflux. The acetonitrile is removed by destillation and toluene is added. It is heated for 14 h under reflux and water separation. The solvent is removed by destination under vacuum and the remainder is recrystallized from dichloremethane. 2.4 g of colorless crystals are obtained.

Mp. 110° C. ¹H-NMR: (d4-MeOH): δ (ppm)=8.08-7.48 (m, 5H, Ar—H), 4.61-4.54 (m, 1H, H-3), 4.50-4.42 (m, 3H, 2×H-6, H-4), 4.22-4.19 (m, 1H, H-5).

### EXAMPLE 5

### 1-(4,4-Difluoro-tetrahydro-3-hydroxy-5-oxo-2-furyl)-1,2-ethanediol-1,2,3-tribenzoate (IX)

2.13 g of benzoyl chloride in ethyl acetate were added dropwise to a solution of 1.95 g of 1-(4,4-Difluoro-tetrahydro-3-hydroxy-5-oxo-2-furyl)-1,2-ethane-diol-2-benzoate (VIII), 1.95 g of pyridine and 0.16 g of dimethylaminopyridine (DMAP) in ethyl acetate at reflux temperature. After 3 further hours of heating under reflux, the reaction mixture is cooled down to room temperature, diluted with ethyl acetate and extracted with water. The aqueous phase is re-extracted with ethyl acetate, the combined organic phases are successively extracted with diluted 1N HCl-solution and with saturated sodium-bicarbonate solution, dried and evaporated. 3 g of oil are obtained.

¹H-NMR: (CDCl₃): δ (ppm)=8.04-7.39 (m, 15H, 3×Ar—H), 5.99-5.98 (m, 1H, H-5), 5.76-5.73 (m, 1H, H-3), 5.15-5.14 (s, 1H, H-4), 4.79-4.76 (m, 2H, 2×H-6)

### EXAMPLE 6

### 5-{[1,2-Bis(benzoyloxy)]ethyl}-3,3-difluoro-tetrahydrofuran-2,4-diol-4-benzoate (X)

2.1 g of lithium aluminum tritertiary butyl aluminum hydride (1M solution in THF) are added dropwise to a solution of 3 g of 1-(4,4-Difluoro-tetrahydro-3-hydroxy-5-oxo-2-furyl)-1,2-ethanediol-1,2,3′-tribenzoate (IX) in 10 ml of THF and 40 ml of diethyl ether at 0° C. After two hours of stirring 1N HCl is acidified under cooling. The solution is diluted with MTBE and the phases are separated. The aqueous phase is once again extracted with MTBE, the combined organic phases are neutralized with 5% NaHCO₃-solution, dried and evaporated. 2.71 g of light-yellow oil are obtained.

¹H-NMR: (CDCl₃): δ (ppm)=7.61-7.26 (m, 15H, 3×Ar—H), 6.01-4.95/5.47-5.43 (m, 1H, H-3), 5.93-5.91/5.70-5.67 (m, 1H, H-5), 5.42/5.28 (d, 1H, H-1), 4.79-4.58 (m, 3H, H-4, 2×H-6).

### EXAMPLE 7

### 5-{[1,2-Bis(benzoyloxy)]ethyl}-3,3-difluoro-tetrahydrofuran-2,4-diol-2-methansulfonate-4-benzoate (XI)

0.98 g of triethyl amine and, subsequently, 0.89 g of methanesulfonyl chloride are slowly added to a solution of 3.3 g of 5-{[1,2-Bis(benzoyloxy)]ethyl}-3,3-difluoro-tetrahydrofuran-2,4-diol-4-benzoate (X) in anhydrous dichloromethane at 0° C. and the solution is first stirred for 30 minutes under cooling and then for 2 hours at room temperature. The reaction mixture is diluted with dichloromethane and successively extracted with 1N HCl and 5% NaHCO₃-solution. The organic phase is dried and evaporated. 3.44 g of oily product are obtained.

¹H-NMR: (CDCl₃): δ (ppm)=8.14-7.34 (m, 15H, 3×Ar—H), 6.19-6.18/6.08-6.06 (d, 1H, H-1), 5.99-5.79 (m, 2H, H-3, H-5), 5.75-5.73 (q, 1H, H-1), 5.57-5.47 (m, 1H, H-3), 4.93-4.92/4.79-4.76 (m, 1H, H-4), 4.69/4.64 (m, 2H, 2×H-6), 3.16/3.15 (s, 3H, CH₃).

### EXAMPLE 8

### 4-Amino-1-{5-{[1,2-Bis(benzoyloxy)]ethyl}-4-(benzoyloxy)-3,3-difluoro-tetrahydro-2-furanyl}-2(1H)-pyrimidinone (XII) and 4-Acetylamino-1-{5-{[1,2-Bis(benzoyloxy)]-ethyl}-4-(benzoyloxy)-3,3-difluoro-tetrahydro-2-furanyl}-2(1H)-pyrimidinone (XIIa)

### a) Bis(trimethylsilyl)-N-acetylcytosine

A suspension of N-acetylcytosine, hexamethyldisilazane and ammonium sulfate is heated for 5 hours under reflux. Subsequently, the excessive hexamethyldisilazane is removed by destination and the raw product is distilled. 0.7 g of product are obtained as a light-yellow oil, Bp.: 150° C./0.16 mm of Hg.

### b) Nucleosidation

A solution of 0.54 g of Bis(trimethylsilyl)-N-acetylcytosine in dichloroethane is stirred with 0.41 g of trifluoromethane sulfonic acid trimethylsilyl ester for 1 hour at room temperature, 0.72 g of 5-{[1,2-Bis(benzoyloxy)]ethyl}-3,3-difluoro-tetrahydrofuran-2,4-diol-2-methane-sulfonate-4-benzoate (XI) are added and the solution is heated for 16 h under reflux. The reaction mixture is diluted with dichloroethane and first extracted with water and then with 5%-sodium bicarbonate solution. The organic phase is dried and evaporated. The raw product is separated over VFC. 48 mg of 4-Acetylamino-1-{5-{[1,2-Bis(benzoyloxy)]-ethyl}-4-(benzoyloxy)-3,3-difluoro-tetrahydro-2-furanyl}-2(1H)-pyrimidinone and 287 mg of 4-Amino-1-{5-{[1,2-Bis(benzoyloxy)]ethyl}-4-(benzoyloxy)-3,3-difluoro-tetrahydro-2-furanyl}-2(1H)-pyrimidinone are obtained as anomer mixture. The mixture can also be used for the next reaction in an unseparated form.

¹H-NMR (anomer mixture of XII): (CDCl₃): δ (ppm)=8.10-7.85 (m, 6H, 3×benzoyl-H-2,6); 7.62-7.35 (m, 10H, Benzoyl-H-3,4,5, H-6), 6.77-6.59 (m, 1H, H-1′), 5.94-5.92/5.89-5.86 (m, 2H, H-5, H-5′), 5.82-5.77/5.67 (m, 1H, H-3′), 4.86 (m, 1H, H-4′), 4.78-4.64 (m, 3H, H-4′, 2×H-6′), see also FIGS. 1a, 1b and 1c.

### EXAMPLE 9

### 4-Amino-1-{5-[(1,2-dihydroxy)]ethyl]-3,3-difluoro-tetrahydro-4-hydroxy-2-furanyl}-2(1H)-pyrimidinone (XIII) and 4-Acetylamino-1-{5-[(1,2-dihydroxy)]ethyl]-3,3-difluoro-tetrahydro-4-hydroxy-2-furanyl}-2(1H)-pyrimidinone (XIIIa)

87 mg of a mixture of 4-Amino-1-{5-{[1,2-Bis(benzoyloxy)]ethyl}-4-(benzoyloxy)-3,3-difluoro-tetrahydro-2-furanyl}-2(1H)-pyrimidinone (XII) and 4-Acetylamino-1-{5-{[1,2-Bis(benzoyloxy)]-ethyl}-4-(benzoyloxy)-3,3-difluoro-tetrahydro-2-furanyl}-2(1H)-pyrimidinone (XIIa) are stirred with 7N NH₃ in methanol for 16 hours at room temperature and are subsequently evaporated to dryness. The remainder is taken up in water and extracted with diethyl ether. The aqueous phase is evaporated in vacuum. 49 mg of light-brown oil are obtained.

¹H-NMR (Anomer mixture): (MeOD): δ (ppm)=8.00-7.98/7.63-7.61 (m, 1H, H-6), 6.39-6.36/6.20-6.17 (m, 1H, H-1′), 5.96-5.90 (m, 1H, H-5), 4.58-4.52/4.36-4.30 (m, 1H, H-3′), 4.29-4.26/3.99-3.97 (m, 1H, H-4′), 3.86-3.83/3.76-3.73 (m, 1H, H-5′), 3.72-3.65 (m, 2H, 2×H-6′), see also FIG. 2

### EXAMPLE 10

### Gemcitabine×HCl

Under ice-cooling a solution of 0.1 g of sodium periodate in water is added dropwise to a solution of 0.08 g of a mixture of 4-Amino-1-{5-[(1,2-dihydroxy)]ethyl]-3,3-difluoro-tetrahydro-4-hydroxy-2-furanyl}-2(1H)-pyrimidinone (XIII) and 4-Acetylamino-1-{5-[(1,2-dihydroxy)]ethyl]-3,3-difluoro-tetrahydro-4-hydroxy-2-furanyl}-2(1H)-pyrimidinone (XIIIa) in methanol, and the solution is stirred for further 15 min under ice-cooling and then for 1 h at room temperature. Subsequently, 0.02 g of sodium borohydride are added under ice-cooling and after 15 min stirring is continued for 1 h at room temperature. The solid is filtrated off, the filtrate is neutralized with 5N HCL in i-propanole and evaporated to dryness, taken up in DCM/MeOH (4+1) and filtrated over silica gel. 0.06 g of gemcitabine×HCl are obtained. For further purification it is recrystallized from acetone/water.

Mp: (271-76, dec.) ¹H-NMR: (MeOD): δ (ppm)=8.09-8.07/7.68-7.85 (m, 1H, H-6), 6.35-6.32/6.22-6.19 (m, 1H, H-1′), 6.09-6.05 (m, 1H, H-5), 4.46-4.38/4.33-4.25 (m, 1H, H-3′), 3.96-3.93/3.81-3.78 (m, 1H, H-4′), 3.72-3.61 (m, 1H, H-5′).

Now the alternative synthesis of the compound 4-Amino-1-{5-{[1,2-bis(benzoyloxy)]ethyl}-4-(benzoyloxy)-3,3-difluoro-tetrahydro-2-furanyl}-2(1H)-pyrimidinone (XII), cf. example 8, will be described in more detail:

### 1,2-Isopropylidene-3,5,6-Tribenzoyl-allofuranose (XV)

8.04 g of 1,2-Isopropylidene-3,5,6-Tribenzoyl-allofuranose (XV) are dissolved with 0.25 g of DMAP in 90 ml of dichloromethane/pyridine 2:1 v/v. Under argon atmosphere, 6.6 ml of benzoyl chloride are added dropwise and stirred for 24 hours at room temperature. The reaction is quenched with 20 ml of MeOH. The mixture is diluted with water. The organic phase is washed twice with 40 ml of water, dried over Na₂SO₄ and evaporated. The remainder is dissolved in 60 ml of dichloromethane and washed twice with 40 ml of 1N H₂SO₄ and twice with 40 ml of saturated NaHCO₃. The organic phase is dried with Na₂SO₄ and the solvent is removed by rotation. 12.2 g of product are obtained as a white powder.

¹H NMR: (CDCl₃): δ (ppm)=8.01-7.77 (m, 6H, 3×Benzoyl-H-2,6); 7.35-7.20 (m, 9H, 3×Benzoyl-H-3,4,5); 5.82 (d, 1H, H-1); 5.72 (m, 1H, H-2); 5.09 (m, 1H, H-3); 4.95 (m, 1H, H-5); 4.60-4.56 (m, 3H, 2×H-6, H-4); 1.49 and 1.25 (s, 6H, 2×Isopropylidene-CH₃).

### 3,5,6-Tribenzoyl-allofuranose (XVI)

### Ketal Cleavage

1.34 g of 1,2-Isopropylidene-3,5,6-Tribenzoyl-allofuranose (XV) (2.52 mMol) produced according to the above example are dissolved in 10 ml of 0.1N HCl/acetonitrile and stirred for 2.5 hours at 50° C. The solvent is removed by evaporation, the remainder is taken up in dichloromethane and washed with water twice. The organic phase is dried over Na₂SO₄, filtrated off and the solvent is removed by evaporation. The mixture obtained is separated by chromatography (eluent DCM/EtAOAc 3:1). 600 mg of product are obtained as a white solid.

¹H NMR: (CDCl₃): δ (ppm)=8.01-7.91 (m, 6H, 3×Benzoyl-H-2,6); 7.70-7.23 (m, 9H, 3×Benzoyl-H-3,4,5); 5.70-5.61 (m, 1H, H-1); 5.56-5.45 (m, 2H, H-3, H-5); 4.71-4.38 (m, 3H, 2×H-6, H-2); 4.36 (t, 1H, H-4).

### EXAMPLE 11

### 4,5,6-Tribenzoyl-1,2-di(phenoxyacetyl)-allofuranose (XVII)

127 mg of phenoxyacetyl chloride are added dropwise to a solution of 184 mg of 3,5,6-Tribenzyol-allofuranose (XVI) in 4 ml of pyridine under argon atmosphere and it is stirred for 1 hour. The reaction is quenched with 0.5 ml methanol. After addition of 10 ml of toluene, the solution is evaporated in vacuum. The remainder is chromatographed with petroleum ether/ethyl acetate 3:1-1:1 over 15 g of silica gel. 225 mg of yellow oil are obtained.

¹H NMR: (CDCl₃): δ (ppm)=8.03-7.96 (m, 6H, 3×Benzoyl-H-2,6), 7.60-6.40 (m, 19H, 3×Benzoyl-H-3,4,5,2×Phenoxyacetyl-H-2,3,4,5,6), 6.77 (m, 1H, H-1′), 5.92-5.70 (3H, m, H-2′, H-3′, H-5′); 4.70-4.32 (m, 7H, H-4′, 2×Phenoxyacetyl-CH₂, 2×H-6′).

### EXAMPLE 12

### 1-[3,5,6-Tribenzoyl-2-Phenoxyacetyl-allofuranosyl]-N-acetylcytosine (XVIII)

### Nucleosidation

For production of a protected cytosine derivative, 152.5 mg of bistrimethylsilyl acetamide are added to a suspension of 30.5 mg of N-acetylcytosine in 3 ml anhydrous dichloroethane and the solution is heated under argon atmosphere under stirring until completely clear under reflux. Subsequently, a suspension of 200 mg of 3,5,6-Tribenzoyl-1,2-di(phenoxyacetyl)-allofuranose (XVII) in 3 ml of dry dichloroethane are added dropwise to the solution which has been cooled down to 50° C. After dropwise addition of 100 mg of trifluoromethylsilyl trifluoromethane sulfonate, it is stirred for 16 h at 80° C. The reaction mixture is partitioned between dichloromethane and saturated aqueous NaHCO₃-solution. Then, the organic phase is washed with several times water and NaCl-solution. The combined organic phases are dried over Na₂SO₄, filtrated and evaporated on vacuum. The reaction mixture is further purified by means of vacuum flash chromatography (DCM-MeOH 9:1). 185 mg of anomer mixture are obtained. The product can be used for the next reaction without any further purification.

¹H NMR: (CDCl₃): δ (ppm)=9.76 (s, 1H, NH); 8.13-7.85 (m, 6H, 3×Benzoyl-H-2,6); 7.58-6.89 (m, 13H, 3×Benzoyl-H-3,4,5; Phenoxyacetyl-H-2,4,6, H-6); 6.86 (m, 1H, H-1′); 6.74 (m, 2H, Phenoxyacetyl-H-3,5); 6.15-5.80 (m, 4H, H-5, H-5′, H-3′, H-2′); 4.91-4.43 (m, 5H, Phenoxyacetyl-CH₂, H-4′, 2×H-6′); 2.42 (s, 3H, N-Acetyl-CH₃).

### EXAMPLE 13

### 1-[3,5,6-Tribenzoyl-allofuranosyl]-cytosine (XIX)

### Selective Saponification

Under an argon atmosphere 17 mg of hydrazine monohydrate are added as 5%-solution in glacial-acetic-acid pyridine to a solution of 80 mg of 1-[3,5,6-Tribenzoyl-2-Phenoxyacetyl-allofuranosyl]-N-acetyl-cytosine (XVIII) in 1.5 ml of glacial-acetic-acid-pyridine mixture (1:4 v/v). The solution is stirred for 15 hours at 70-75° C. Subsequently, 2 ml of acetone are added dropwise, the reaction mixture is diluted with dichloromethane and several times washed with water. The combined organic phases are dried over sodium sulfate, filtrated and evaporated. After vacuum flash chromatography (eluent: Dichloromethane with 1-4% of MeOH), 53 mg of product are obtained as an oil.

¹H NMR: (CDCl₃): δ (ppm)=8.14-7.96 (m, 6H, 3×Benzoyl-H-2,6), 7.64-7.26 (m, 10H, 3×Benzoyl-H-3,4,5, H-6); 6.91-6.77 (m, 1H, H-1′); 5.89-5.61 (m, 3H, H-5, H-3′, H-5′); 4.97-4.43 (m, 4H, H-2′, H-4′, 2×H-6′).

### EXAMPLE 14

### 4-Amino-1-{5-{[1,2-bis(benzoyloxy)]ethyl}-4-(benzoyloxy)tetrahydro-3-oxo-2-furanyl}-2(1H)-pyrimidinone (XX)

### Oxidation

80 mg of 1-[3,5,6-Tribenzoyl-allofuranosyl]-cytosine (XIX) are mixed with 1.2 mg of TEMPO (2,2,6,6-Tetramethyl-piperidine-1-oxyl) and dissolved in 5 ml of dichloromethane. The solution is cooled down to 0-5° C. on the ice-water bath. 1.8 mg of potassium bromide are dissolved in 0.25 ml of H₂O and dropwise added to the mixture. NaOCL is adjusted to a pH of 9.5 with NaHCO₃ and 0.26 ml thereof are slowly and dropwise added while temperature is controlled. It is stirred for further 10 min on the ice-water bath. The entire reaction is observed by means of DC-control (DCM/MeOH 9:1). The organic phase is evaporated, 70 mg of product are obtained as an oil.

¹H NMR: (CDCl₃): δ (ppm)=8.02-7.82 (m, 6H, 3×Benzoyl-H-2,6); 7.83-7.30 (m, 10H, 3×Benzoyl-3,4,5, H-6, H-5); 6.90-6.59 (m, 1H, H-1′); 6.08-5.56 (m, 3H, H-5, H-5′, H-3′); 4.87-4.71 (m, 3H, 2×H-6′, H-4′).

### EXAMPLE 15

### 4-Amino-1-{5-{[1,2-bis(benzoyloxy)]ethyl}-4-(benzoyloxy)-3,3-difluoro-tetrahydro-2-furanyl}-2(1H)-pyrimidinone (XII)

### Fluoridation

100 mg of 4-Amino-1-{5-{[1,2-bis(benzoyloxy)]ethyl}-4-(benzoyloxy)-tetrahydro-3-oxo-2-furanyl}-2(1H)-pyrimidinone (XX) are dissolved in 1 ml of dichloromethane. It is stirred at room temperature and, subsequently, 20 mg of DAST (diethylaminosulfur trichloride) are added dropwise. After the addition has been completed, pyridine-HF (about 30 μl) is added. The mixture is stirred for 48 hours at room temperature, whereupon 39 mg of product are obtained as an oil.

The ¹H NMR (CDCl₃) of the compound (XII) produced according to example 15 is the same as the compound (XII) produced according to example 8, cf. also FIG. 2.

1.-28. (canceled)

29. A compound of formula 2: 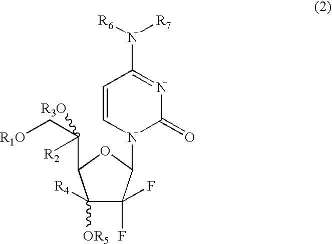
wherein:
R₁, R₃ and R₅ independently represent hydrogen or a suitable hydroxy protective group;
each of R₂ and R₄ represents hydrogen or alkyl with 1 to 6 C-atoms;
each of R₆ and R₇ represents hydrogen or a suitable amino protective group; and
the wavy line in each case represents both possible configurations of —OR₃ and/or —OR₅ with regard to the parent substance.

30. The compound of claim 29, wherein at least one of R₁, R₃ and R₅ is a benzoyl group.

31. The compound of claim 29, wherein R₁, R₃ and R₅ represent hydrogen or a benzoyl group, R₂ and R₄ each represent hydrogen, and each of R₆ and R₇ represents hydrogen, acetyl, alkylsilanyl or arylalkylsilanyl with 1 to 6 C-atoms in the alkyl moiety.

32. The compound of claim 29, wherein each of R₁ to R₇ represents hydrogen.

33. A method of producing a compound of claim 29, comprising reacting a compound of formula 3: 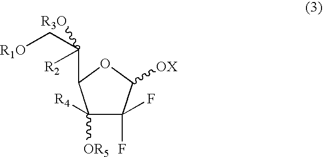
wherein R₁ to R₅ are as defined in claim 29, X represents hydrogen or an activating group known per se, and the wavy line in each case represents both possible configurations of OX, —OR₃ and/or —OR₅ with regard to the parent substance;
with a cytosine having protective groups of formula 4: 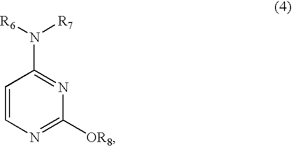
wherein R₆ and R₇ are as defined in claim 29, and R₈ is a suitable leaving group;
wherein, subsequently, any protective groups possibly present are optionally cleaved to obtain a compound of formula (2) wherein each of R₁ to R₇ represents hydrogen.

34. The method of claim 33, wherein X represents an alkylsulphonyl residue with 1 to 6 C-atoms in the alkyl moiety, and at least one of R₆ and R₇ represents trialkylsilanyl or triarylalkylsilanyl with 1 to 6 C-atoms each in the alkyl moiety.

35. The method of claim 33, wherein R₈ is equal to R₆ and R₇.

36. A compound of formula 3: 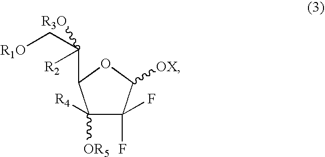
wherein R₁ to R₅ are as defined in claim 29, X represents hydrogen or an activating group known per se, and the wavy line in each case represents both possible configurations of OX, —OR₃ and/or —OR₅ with regard to the parent substance, or the wavy line, together with OX, represents a keto group.

37. The compound of claim 36, wherein R₁ represents a suitable hydroxy protective group, R₂ to R₅ represent hydrogen, and the wavy line, together with OX, represents a keto group.

38. The compound of claim 37, wherein R₁ represents a benzoyl group.

39. The compound of claim 36, wherein R₁, R₃ and R₅ independently represent a suitable hydroxy protective group, and the wavy line, together with OX, represents a keto group.

40. The compound of claim 36, wherein R₁, R₃ and R₅ independently represent a suitable hydroxy protective group, and X represents hydrogen.

41. The compound of claim 40, wherein each of R₁, R₃ and R₅ represent a benzoyl group.

42. The compound of claim 36, wherein R₁, R₃ and R₅ independently represent a suitable hydroxy protective group, and X represents an activating group.

43. The compound of claim 42, wherein each of R₁, R₃ and R₅ represent a benzoyl group, and X represents an alkylsulphonyl residue with 1 to 6 C-atoms in the alkyl moiety.

44. A method of producing a compound of claim 29 comprising:
fluorinating with a suitable fluorinating agent a compound of formula 12: 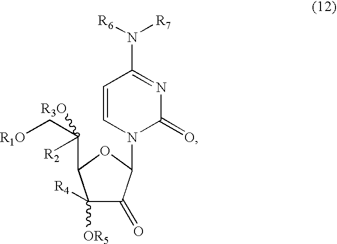
wherein R₁ to R₇ are as defined in claim 29, and the wavy line in each case represents both possible configurations of OX, —OR₃ and/or —OR₅ with regard to the parent substance; and
subsequently, optionally cleaving any protective groups possibly still present to obtain a compound of formula (2), wherein each of R₁ to R₇ represents hydrogen.

45. The method of claim 44, wherein said fluorination is effected with DAST (diethylaminosulfur trichloride) in combination with HF.

46. The method of claim 44, wherein R₁, R₃ and R₅ represent a benzoyl group, and each of R₂, R₄, R₆ and R₇ represents hydrogen.

47. A compound of formula 12: 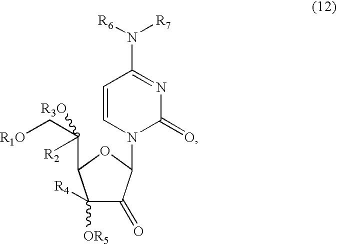
wherein R₁ to R₇ are as defined in claim 29, and the wavy line in each case represents both possible configurations of OX, —OR₃ and/or —OR₅ with regard to the parent substance.

48. The compound of claim 47, wherein R₁, R₃ and R₅ represent a benzoyl group, and each of R₂, R₄, R₆ and R₇ represents hydrogen, and at least one of R₆ and R₇ represents hydrogen, trialkylsilanyl or triarylalkylsilanyl with 1 to 6 C-atoms each in the alkyl moiety.

49. The compound of claim 48, wherein both R₆ and R₇ represent hydrogen.

50. A method for producing gemcitabine, wherein a compound of 29 is optionally deprotected and subjected to a glycol cleavage to the aldehyde of formula 7: 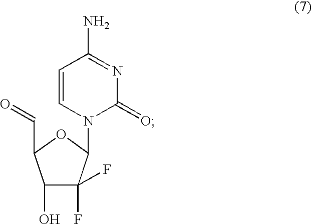
wherein the aldehyde group of the aldehyde 7 is reduced with a complex hydride, thus obtaining gemcitabine of formula 1:

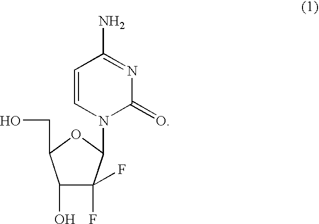

51. The method of claim 50, wherein the glycol cleavage is effected with a periodate.

52. The method of claim 50, wherein reducing is effected with sodium borohydride.

53. The method of claim 50, wherein in the glycol cleavage the pure O-anomer of compound of formula (2), wherein each of R₁ to R₇ represents hydrogen, is used.

54. The method of claim 50, wherein in the glycol cleavage a mixture of the α- and β-anomers of compound of formula (2), wherein each of R₁ to R₇ represents hydrogen, is used.

55. The method of claim 50, wherein said reduction is performed in a one-pot reaction after glycol cleavage.

56. A method of treating a proliferative disease in a subject comprising:
obtaining a compound of claim 29; and
administering the compound to a subject;
wherein a proliferative disease in the subject is treated.

57. The method of claim 56, wherein the proliferative disease is NSCLC (non-small cell lung cancer), mamma carcinoma, ovarian carcinoma, pancreas carcinoma, or bladder carcinoma.

58. The method of claim 56, comprising administering a second active substance/medicament to the subject.

59. The method of claim 56, wherein the subject is a human.
